(19) United States
(12) Patent Application Publication
(10) Pub. No.: US 2015/0121808 A1
(43) Pub. Date: May 7, 2015

(54) BLOOD CLEANSING SYSTEM

(71) Applicants: Angelo Gaitas, Miami, FL (US); Gwangseong Kim, Miami, FL (US)

(72) Inventors: Angelo Gaitas, Miami, FL (US); Gwangseong Kim, Miami, FL (US)

(21) Appl. No.: 14/564,042

(22) Filed: Dec. 8, 2014

Related U.S. Application Data
(63) Continuation-in-part of application No. 14/482,270, filed on Sep. 10, 2014.
(60) Provisional application No. 61/900,070, filed on Nov. 5, 2013.

(51) Int. Cl. A61M 1/36

(52) U.S. Cl. CPC A61M 1/362; A61M 1/3679
USPC 53/431

(57) ABSTRACT

The present invention relates to removing disease material from the blood of a patient. Specifically, the invention relates to using biological binders to trap disease material that is desired to be removed from the blood of a patient.

### CROSS REFERENCE TO RELATED APPLICATIONS

This is a continuation in part of application Ser. No. 14/482,270 filed Sep. 10, 2014, which claims the benefit of U.S. Provisional Patent Application No. 61/900,070 filed Nov. 5, 2013 and entitled “A Blood Cleansing System,” the entire disclosures of which are incorporated herein by reference.

### GOVERNMENT SUPPORT

This invention was made with government support under U.S. Public Health Service Grant No. GM084520 from the National Institutes of Health. The Government has certain rights in the invention.

### FIELD OF THE INVENTION

The present invention relates to removing disease material from the blood of a patient. Specifically, the invention relates to using biological binders to trap disease material that is desired to be removed from the blood of a patient.

### BACKGROUND OF THE INVENTION

Many diseases, as well as other harmful particles and biological molecules, are carried by the blood. While there are certain methods directed towards filtering toxins from the blood, existing systems and methods do not target specific particles for removal from the blood. In general, for cell capturing, a cell surface marker is targeted, such as a protein or receptor on the membrane, using an antibody or aptamer linked to a device surface. However, there are no existing methods that utilize the previously mentioned capture technique to target and remove particles from the blood.

Therefore there is a need in the art for a system and method to remove unwanted particles, cells, and bio-molecules from blood by targeting specific particles. These and other features and advantages of the present invention will be explained and will become obvious to one skilled in the art through the summary of the invention that follows.

### SUMMARY OF THE INVENTION

Accordingly, it is an object of the present invention to provide a method for removing disease material from the blood of a patient. In one embodiment this invention is used to reduce metastatic cancer. In cancer metastasis cells from a primary tumor become circulating tumor cells (CTCs) and then adhere to other organs to create a metastasis. This invention discloses a method and an apparatus to remove cancer cells from the blood of a patient in order to reduce or minimize metastasis. This invention can also be used to remove viruses, microorganisms, bacteria, metastatic cells, materials, peptides such as beta amyloid (Amyloid beta (Aβ or Abeta) is a peptide of 36-43 amino acids that is processed from the amyloid precursor protein (APP)) that play a critical role in diseases such as Alzheimer's, proteins, enzymes, toxins, diseased cells, and cancer cells. This invention can help reduce infections including, but not limited to sepsis and high lactate level.

According to an embodiment of the present invention, the invention can utilize biological binders such as antibodies to trap microorganisms, cells, cancer cells, circulating tumor cells, peptides, and other material that is desired to be removed from blood.

According to an embodiment of the present invention, a patient's blood is pumped and flown though an apparatus that contains a filter or filters or a device with pillars (or micropillars), micro-posts, tube or tubes, well(s) with a microfluidic reaction chamber (made of a spiraling microfluidic tube), microspheres (beads or microbeads) or spheres, or any combination thereof. Biological binders have been pre-coated on the apparatus or on parts of the apparatus such as the microspheres. Alternatively, the apparatus may include a mechanism for size separation. In some embodiments, the apparatus may include a semi-permeable membrane. In a preferred embodiment, as blood flows through the apparatus, undesired substances are trapped (for example CTCs) while red blood cells and desired substances are re-circulated back into the patient. The process can be repeated several times. In some embodiments, the trapped substances are further analyzed to examine and study disease progression.

According to an embodiment of the present invention, a method for removing disease causing material from blood includes the steps of: pumping blood from a patient into a cleansing apparatus; flowing said blood through said cleansing apparatus to expose said blood to a binding material; capturing disease causing material, wherein said binding material targets and binds to said disease causing material; removing said disease causing material from said blood; and returning said blood to said patient.

According to an embodiment of the present invention, the blood is pumped to said cleansing apparatus until said cleansing apparatus is full thereby allowing said binding material to capture said disease causing material.

According to an embodiment of the present invention, the binding material is one or more binding materials selected from a group of binding materials comprising antibodies, peptides, proteins, aptamers, TNF-related apoptosis-inducing ligands (TRAIL), ligands, apoptosis inducing substances, death receptors binding substances, tumor necrosis factors, adhesion receptors, E-selectin, cytokines, chemotherapy agents, biological binders.

According to an embodiment of the present invention, the method further includes the step of analyzing said disease causing material that has been captured by said binding material.

According to an embodiment of the present invention, the method further includes the step of counting the amount of said disease causing material trapped in said cleansing apparatus.

According to an embodiment of the present invention, the disease causing material is one or more disease causing materials selected from a group of disease causing materials comprising cancer stem cells, metastatic cancer cells, cancer cells, circulating tumor cells, viruses, microorganisms, bacteria, peptides, beta amyloid, proteins, enzymes, toxins, diseased cells, cancer cells, enzymes, toxins, diseased cells, infectious microorganisms, cells, disease cells, fungi.

According to an embodiment of the present invention, the cleansing apparatus is comprised of an inlet, an outlet, and a cleaning mechanism for removing said disease causing material.

According to an embodiment of the present invention, an inner surface of said cleansing apparatus is coated with said binding material.

According to an embodiment of the present invention, the cleansing mechanism is comprised of a plurality of spheres, each of has an outer surface that is coated with said binding material.

According to an embodiment of the present invention, the cleansing mechanism is comprised of a plurality of pillars, each of which is coated with said binding material.

According to an embodiment of the present invention, the cleansing mechanism is comprised or one or more tubes, each of which has an inner surface that is coated with said binding material.

According to an embodiment of the present invention, the cleansing mechanism is further comprised of a nanorough surface.

According to an embodiment of the present invention, the cleansing mechanism is further comprised of a microrough surface.

The foregoing summary of the present invention with the preferred embodiments should not be construed to limit the scope of the invention. It should be understood and obvious to one skilled in the art that the embodiments of the invention thus described may be further modified without departing from the spirit and scope of the invention.

### BRIEF DESCRIPTION OF THE DRAWINGS

FIG. 1 is an illustration of a patient's blood being pumped and flown through the cleansing device, after which the cleansed blood is injected back into the patient.

FIG. 2 is an illustration of a patient's blood being pumped and flown through the cleansing device, after which the cleansed blood is injected back into the patient.

FIG. 3 is an illustration a pressure monitor, a heparin pump, and an inflow pressure monitor in accordance with an embodiment of the present invention.

FIG. 4 is an illustration of blood flowing from the patient through a tube to a cleansing device with spheres that include a binding material.

FIG. 5 is an illustration of a capturing device including pillars coated with binding material, in accordance with an embodiment of the present invention.

FIG. 6 is an illustration of a capturing device composed of tube(s) coated with binding material, in accordance with an embodiment of the present invention.

FIG. 7 is an illustration of a device that uses filtering to separate wanted from unwanted material in the blood, in accordance with an embodiment of the present invention.

FIG. 8 is an illustration of a tube with captured material for removal, in accordance with an embodiment of the present invention.

FIG. 9 is an illustration of a light or radiation exposure unit included on the device to achieve photochemotherapy or radiotherapy.

FIG. 10 shows the steps of a tube coating process, in accordance with an embodiment of the present invention.

FIG. 11 shows the steps of a tube coating process.

FIG. 12 contains pictures of actual tubes with fluorescently labeled captured cells, in accordance with an embodiment of the present invention.

FIG. 13 shows the steps of a tube coating process, in accordance with an embodiment of the present invention.

### DETAILED SPECIFICATION

The present invention relates to removing disease material from the blood of a patient. Specifically, the invention relates to using biological binders to trap disease material that is desired to be removed from the blood of a patient.

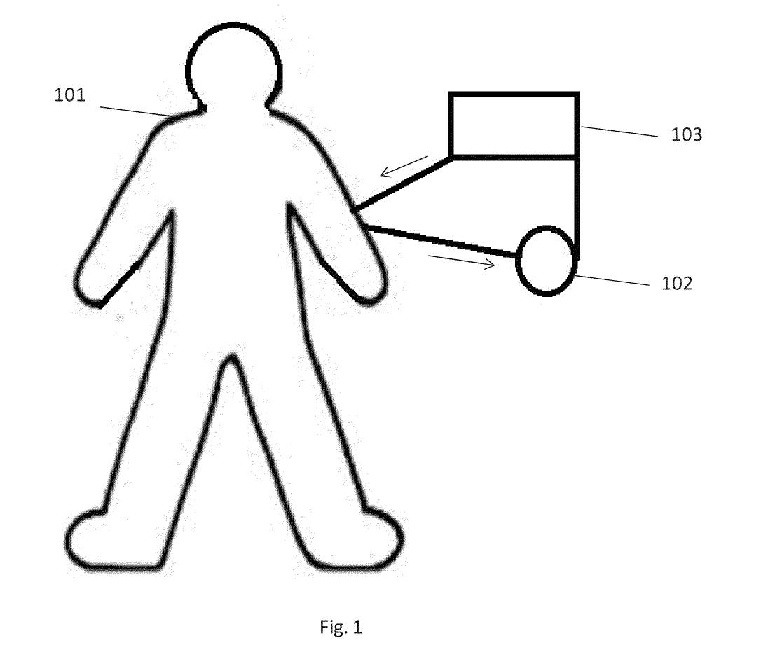

According to an embodiment of the present invention, as shown in FIG. 1, the patient's (101) blood is moved by a pump (102) and flown through the cleansing device (103). After the cleansing process is complete, the patient's blood is injected back in the patient.

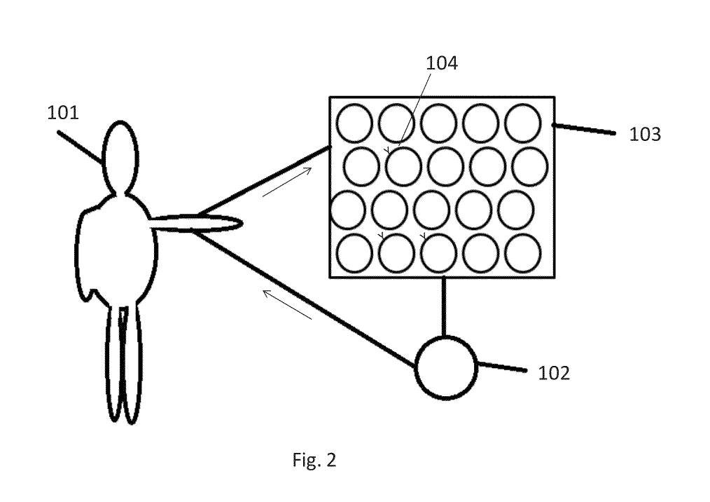

According to an embodiment of the present invention, as shown in FIG. 2, the patient's (101) blood is moved by a pumped (102) and flown through the cleansing device (103). After the cleansing process is complete, the patient's blood is injected back in the patient. In a preferred embodiment, the cleansing device (103) contains spheres with specific biological binders, such as antibodies (104), to that target and bind to the specific particles that are desired to be removed from the patient's blood.

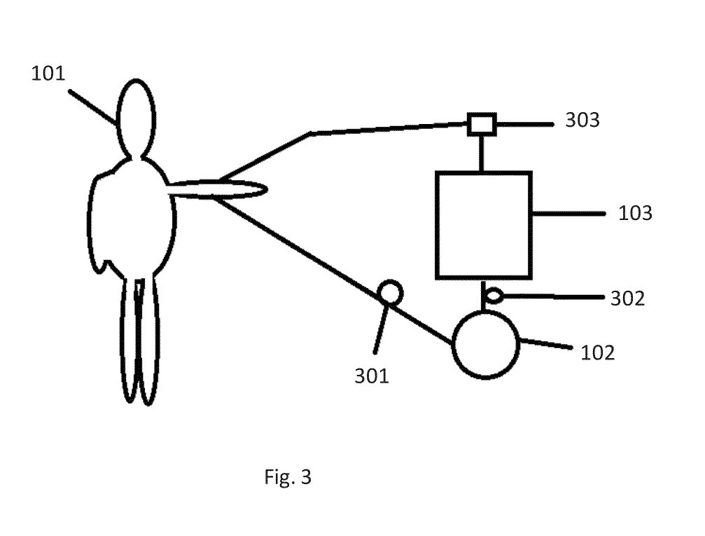

According to an embodiment of the present invention, as shown in FIG. 3, a pressure monitor (301) may be used to measure arterial pressure. In some embodiments, a heparin pump (302) and an inflow pressure monitor may also be included. In some embodiments, a venous pressure monitor and/or an air trap and air detector (303) are also included. Certain embodiments of the present invention may include fewer or additional components and the present invention may be used with any combination of the mentioned and additional components to achieve the desired functionality. One of ordinary skill in the art would appreciate that the cleansing device may be configured with any number of components based upon the desired functionality for the cleansing device, and embodiments of the present invention are contemplated for use with any such component.

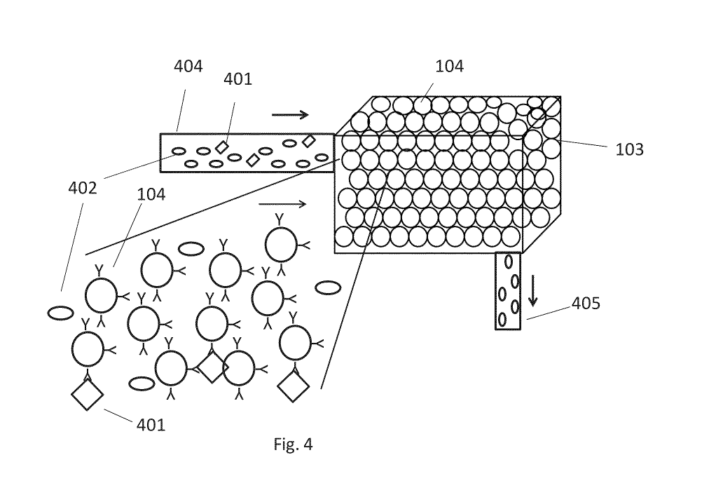

According to an embodiment of the present invention, as shown in FIG. 4, blood flows from the patient through a tube to the cleansing device (103). In the preferred embodiment, the cleansing device (103) includes spheres with binding material (104). In some embodiments, the binding materials are antibodies or aptamers specific to the cell surface marker of the cells that are being targeted for removal, such as circulating tumor cells (CTCs) (401). CTCs detach from both primary and metastatic lesions and attach to other areas on the body. As unwanted material (401) such as CTCs flow through the device, (103) they are captured and removed (as shown in FIG. 4). The resulting output blood is clean of unwanted material and is returned to the body of the patient. In some embodiments, the surface of the cleansing device (103) or of the sphere (104) (or of the tube or of the pillar) is a nanorough surface that captures cells such as CTCs. A nanorough surface possesses nanometer scale roughness. A microrough surface possesses micrometer scale roughness. One of ordinary skill in the art would appreciate that the cleansing device could be used with any binding material, and embodiments of the present invention are contemplated for use to target and remove any cell type.

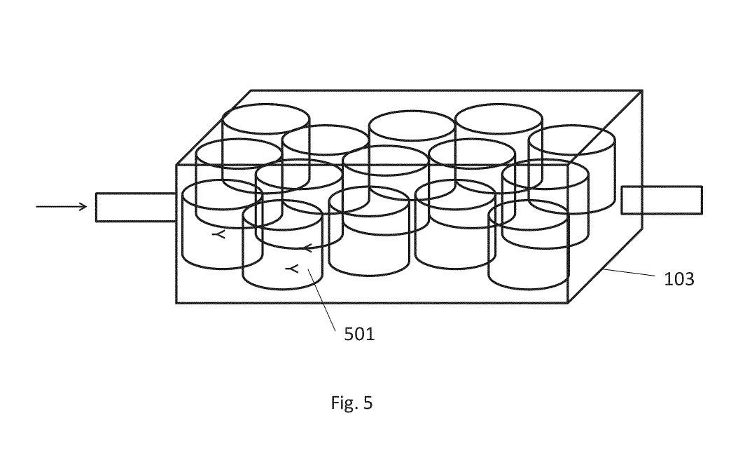

According to an embodiment of the present invention, in FIG. 5, the cleansing device (103) includes pillars (501) coated with binding material. In a preferred embodiment, the pillars are tightly positioned to increase the chances that the desired particles will collide and stick to the pillars. One of ordinary skill in the art would appreciate that there would be many useful patterns and arrangements that the pillars could be positioned in, and embodiments of the present invention are contemplated for use with any such arrangement.

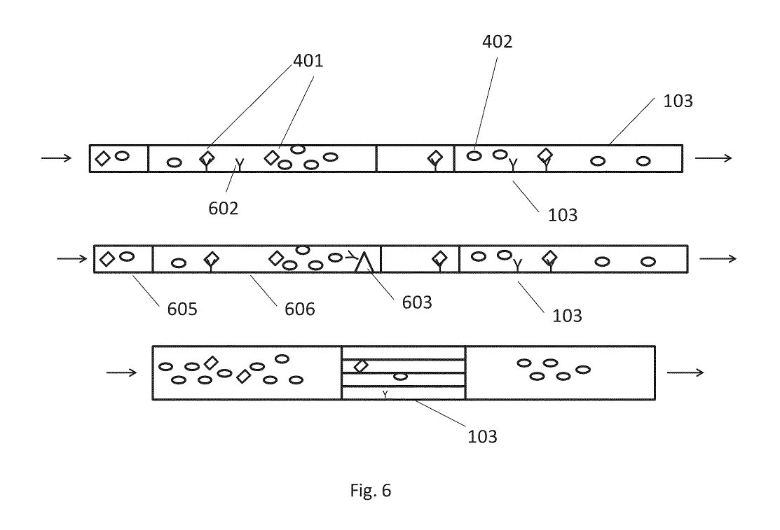

According to an embodiment of the present invention, as shown in FIG. 6, the cleansing device is composed of tubes (103), for example flexible tubes, coated with binding material (603) such as adhesion protein. In some embodiments the flexible tube includes a nanorough or microrough surface. In some embodiments, multiple tubes join together (for example 605 and 606), with each tube having different binding materials (602), such as different antibodies for separate diseases. In a preferred embodiment, this allows the cleansing device to target and remove multiple types of cell types from the blood. In a preferred embodiment, as blood flows out of the patient and into the cleansing device, the blood passes from each tube trapping unwanted disease causing material such as cancer cells. In some embodiments, as shown in FIG. 1, a pump is used to move the blood through the cleansing device. Ultimately, the cleaned blood is returned to the patient. In some embodiments, the tubes are pre-coated with a binding material. In some embodiments the tubes are coated by flowing various chemicals and biomolecules including binding agents through the tubes before connecting the device to the patient. In some embodiments the tubes include barriers (constriction areas) (603) to make cells and flowing material collide with the tube walls or barriers in order to increase the probability of capture. According to an embodiment of the present invention, the tubes are flexible. In a preferred embodiment, the tubes are spiral or otherwise meandering in shape. In alternate embodiments, the tubes may be rigid and straight in shape. One of ordinary skill in the art would appreciate there any many suitable designs for a tube, and embodiments of the present invention are contemplated for use with any such tube design.

According to an embodiment of the present invention, after treatment is completed, the tube or tubes can be used to analyze the remaining cells via florescent tagging or imaging or other techniques such as cytometry. Similarly ELISA, fluorogenic, electrochemiluminescent, or chromogenic reporters or substrates that generate visible color change to pinpoint the existence of antigen or analyte may be used to analyze the sample. In some embodiments, heat treatment of blood may also be performed. For example, applying heat of a specific temperature may be useful to destroy unwanted cells or other material. In some embodiments, medications, drugs, chemicals or any combination thereof may be added to attack the unwanted material, such as cancer cell, bacteria, viruses, or other biomolecules. In some embodiments, the drugs are removed before the blood is returned to the body. In a preferred embodiment, the drug removal is done by filtering or other methods like the ones described in this disclosure. In some embodiments, radiation may also be used in the cleansing process. Various types of cancer including leukemia are addressed this way and the clean blood is reinserted in the patient. In some embodiments, (arrangement shown at the bottom of FIG. 6) multiple micro-tubes are used. As previously these micro-tubes are functionalized with binding (capturing) material (602). The micron size of the tube (for example 20 micron, or 10 micron, or 30 micron, or 50 micron, or 100 micron or 500 micron or less than 2 mm) increases the capturing possibility, while the large number of the micron size tubes in parallel does not hinder the throughput enabling fast flow.

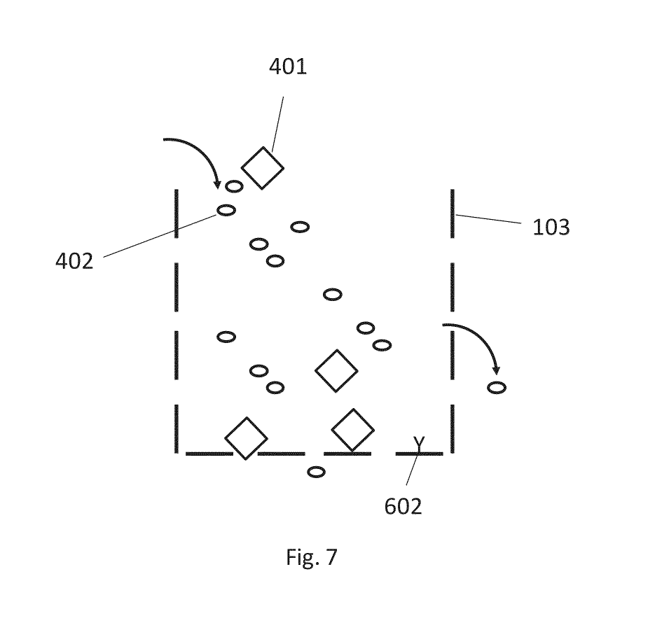

According to an embodiment of the present invention, as shown in FIG. 7, a device that uses filtering is used to separate wanted (402) from unwanted material in the blood. As in illustrative example, CTCs are larger than blood cells. In some embodiments, a binding biomolecule (602) such as an antibody is coated on the walls of the device or on the filter so that the unwanted (401) particle is captured. In some embodiments osmosis is used (much like in dialysis). In some embodiments the filter is made of microfabricate material, including, but not limited to PDMS or other material like polyimide with micron size holes (e.g. example 10 micron size holes). In some embodiments the blood is cleaned and then returned to the patient. In another embodiment blood is transfused to the patient. Alternatively, blood is mixed with functionalized microbeads with conjugated antibodies or binding material. In some embodiments several beads with different binding material such as antibodies are included. In the preferred embodiment, the cells or material that are to be removed bind to the functionalized beads. As the cells flow, the cells are trapped by the filter because the cells are larger than the opening in the filter. In some embodiments, blood is mixed with the beads in a separate container and then the mixture is inserted in the device. As an illustrative example, CTCs are larger than other cells in the blood such as leukocytes, erythrocytes, thrombocytes. For instance, CTCs may have diameters 12-25 microns, therefore a 10 micron opening in the filter may block CTCs from going through, while allowing blood cells, which are 90% smaller, to pass through. In some embodiments centrifugation is used to separate cells with the centrifugal force based on density. Alternatively, hydrodynamic sorting is used. One of ordinary skill in the art would appreciate that many filtering methods exist to enhance the removal of unwanted material form the blood, and embodiments of the present invention are contemplated for use with any such filtering method or any combination thereof.

CTCs are captured using specific antibodies able to recognize specific tumor markers such as EpCAM. In some embodiments of the present invention the spheres, tubes, pillar, filters, or walls (or any combination thereof) of the device are coated with a polymer layer carrying biotin analogues and conjugated with antibodies anti EpCAM for capturing CTCs. After capture and completion, therapy images can be taken to further diagnose disease progression by staining with specific fluorescent antibody conjugates. Antibodies for CTC capture include, but are not limited to, EpCAM, Her2, PSA.

According to an embodiment of the present invention, as shown in FIG. 6, the capturing device is composed of tubes (103), for example flexible tubes, coated with binding material (603) such as adhesion protein. The flexible tube is made of a material selected from the group of materials consisting of, but not limited to, plastic, PDMS, SU-8, polyimide, paralyne, metals, iron, iron oxides, or other materials. In some embodiments, the inner surface of the tube is modified to be receptive to the biological binder, for example to a specific antibody or peptide coating. In some embodiments, the capturing device (such as a simple tube) is coated with peptides. In some embodiments, the patient's blood flows through the capturing device (such as a simple tube), but then flow is stopped so that the relevant biological microorganism, cell, protein, antibody, or peptide is allowed to adhere to the biological binder on the surface of the device. Next, the blood is flown out of the capturing device (such as a simple tube) after given enough time to maximize capturing. In a preferred embodiment, the blood may be flown back out of the capturing device after thirty (30) to sixty (60) minutes. In alternate embodiments, the blood may be flown back out the device after a longer or shorter period depending upon the amount of time required to collect the unwanted material. One of ordinary skill in the art would appreciate this amount could be adjusted accordingly based on the particular application. In some embodiments the tube has a spiral shape, while in others the tube has a stacked spiral shape. One of ordinary skill in the art would appreciate that there are many suitable shapes for a tube, and embodiments of the present invention are contemplated for use with any such tube shape.

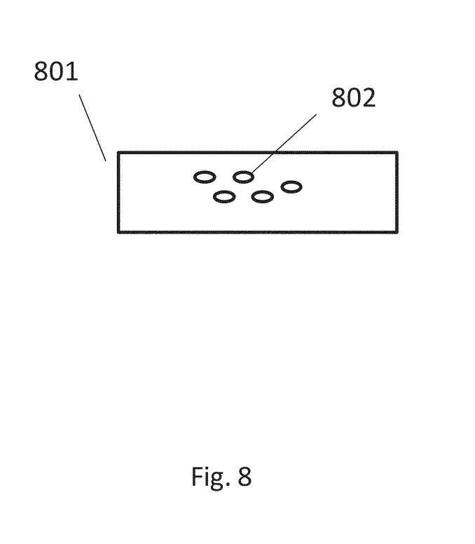

According to an embodiment of the present invention, as shown in FIG. 8, a device 801 with captured material 802 (such as cancer cells) are previously fluorescently tagged with florescent die. For example, FITC labeled antibody is used to tag the cells that have been captured in the device. Next, the florescent cells are counted. In some embodiments an automated system is used to count the cells. The system may include a software system and CCD camera to count the cells. In some embodiments, the entire device is counted. For example, the florescent cells attached to the inner part of the tube are counted by examining the tube outer part. The tube may be rotated to enumerate the cells on all the sides of the tube. In some embodiments, an area is counted and the total number of cells captured is extrapolated from the cell count. In some embodiments the counting is conducted after the capture is completed and the rest of the fluids such as whole blood are removed. One of ordinary skill in the art would appreciate that there are numerous methods to tag and count the cells that are captured, and embodiments of the present invention are contemplated for use with any such method.

According to a first preferred embodiment of the present invention, there is continuous flow through the device. In an alternate preferred embodiment, the device is filled with blood and the flow is stopped for a specific time (for example for 30 minutes), then flow is resumed until the device is full again and the step is repeated.

According to an embodiment of the present invention, the capturing device is exposed to radiation for radiation therapy in order to kill cancer cells or other materials and cells that are malignant. In some embodiments, chemotherapy agents are coated on the surface of the device. As cells flow through the device they collide with the surface of the device and die or attach and die if antibody capturing is also used in combination with chemotherapy agents. In some embodiments chemical substances, such as one or more anti-cancer drugs, are used. In some embodiments, drugs that are not indiscriminately cytotoxic (such as monoclonal antibodies) are coated on the surface of the device. These drugs target specific proteins expressed specifically on the cells that have to be removed, such as proteins on a bacterium or cancer cell.

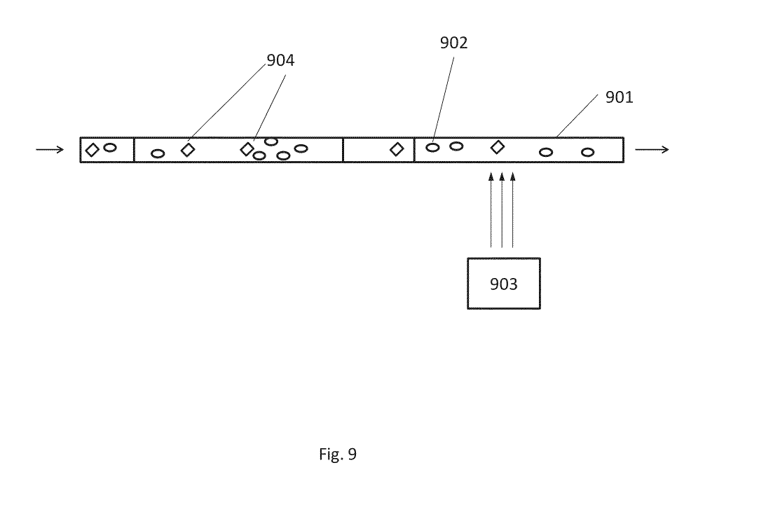

According to an embodiment of the present invention, as shown in FIG. 9, light exposure 903 is included in a way such that the device 901 is exposed to light to achieve photochemotherapy (also referred to as photodynamic therapy). In a preferred embodiment, the target material 904 is destroyed by administering a photosensitizer material intravenously. A nontoxic photosensitizer is typically a light-sensitive compound that becomes toxic when exposed to light. In the preferred embodiment, the photosensitizer is linked to an antibody or peptide that attaches selectively to the target material and the target material flows along with the blood through the device. Light is then delivered to the target material as it passes through the device to cause the destruction of the target material. Photosensitizers are functionalized to specifically attach to the above mentioned targets. Examples of photosensitizers include, but are not limited to, chlorophylls, porphyrins, dyes, Silicon Phthalocyanine Pc 4, aminolevulinic acid, mono-L-aspartyl chlorine, m-tetrahydroxyphenylchlorin (mTHPC). In some embodiments the photosensitizer is linked to an antibody or peptide that is attached to the inner walls of the device (such as the inner tube). The target material 904 flows along with blood 902 through the device 901. Then, the target material attaches to the antibody or peptide linked to the photosensitizer. Light is then delivered to the target material to cause the destruction of the target material.

According to an embodiment of the present invention, this method may be used to target and remove any number of particles from the blood, such as cancer cells, disease cells, viruses (for example HIV and Methicillin-resistant Staphylococcus aureus), microbial species, peptides and proteins that contribute to diseases, pathogens, microbial cells, fungi, bacteria, sepsis causing organisms, toxins, and microorganisms. Furthermore, this method may be used to treat septic shock and sepsis infections caused by bacteria, virus or fungus specifically bloodstream infection (bacteremia). In a preferred embodiment, the blood is decontaminated are returned to the body.

According to an embodiment of the present invention, hyperthermia therapy may be used to aid in the cleansing of the blood. In a preferred embodiment, once blood is flown through the device it is heated to high enough temperatures so as to cause apoptosis or cell death or otherwise destroy or deactivate the target. In the preferred embodiment, heating can be conducted in active flow or without blood flow (e.g. the device is filled with blood, the flow is stopped, and then the device is heated). In some embodiments the device is the cooled to normal body temperatures. In some embodiments there are several chambers (compartments) for cooling and heating.

According to an embodiment of the present invention, the device is coated with a coating, wherein the coating is selected from the group of coatings comprising proteins, antibodies, peptides, TNF-related apoptosis-inducing ligands (TRAIL), ligands, substances that induce apoptosis, substances that binding to certain death receptors, tumor necrosis factors (or the TNF family), adhesion receptors, E-selectin, and cytokines. One of ordinary skill in the art would appreciate there are numerous coatings that might be used and embodiments of the present invention are contemplated for use with any such coating.

According to an embodiment of the present invention, this invention may also be used to remove viruses, microorganisms, bacteria, metastatic cells, materials, cancer stem cells (CSCs), or peptides (e.g. beta amyloid (Amyloid beta (Aβ or Abeta) is a peptide of 36-43 amino acids that is processed from the amyloid precursor protein (APP)) that play a critical role in diseases such as Alzheimer's), proteins, enzymes, toxins, diseased cells, cancer cells. In a preferred embodiment, this invention can help reduce infections including sepsis and high lactate level. The invention may utilize biological binders such as antibodies or peptides to trap microorganisms, bacteria, viruses, infectious microorganisms, cells, cancer cells, circulating tumor cells, peptides, and other material that are desired to be removed from blood.

According to an embodiment of the present invention, an extracorporeal filtration device may be used to remove CTCs from the bloodstream aiming at reducing the chances of metastasis by modifying a commercially available plastic tube that is functionalized with EpCAM antibodies. In a preferred embodiment, blood flows through a tube where CTCs bind to EpCAM antibodies coated on the inner surface of the tube. In the preferred embodiment, this procedure can be done safely and successfully in a clinical setting by (i) processing the entire blood in continuous circulation or (ii) consecutive drawing of as much as 0.5 liter of blood (a quantity in line with typical blood donations), undergoing the cleaning process for CTC removal, and re-injecting the blood in the patient, then repeating the process until all of the blood is cleaned from CTCs (a typical adult has a blood volume between 4.7 and 5 liters).

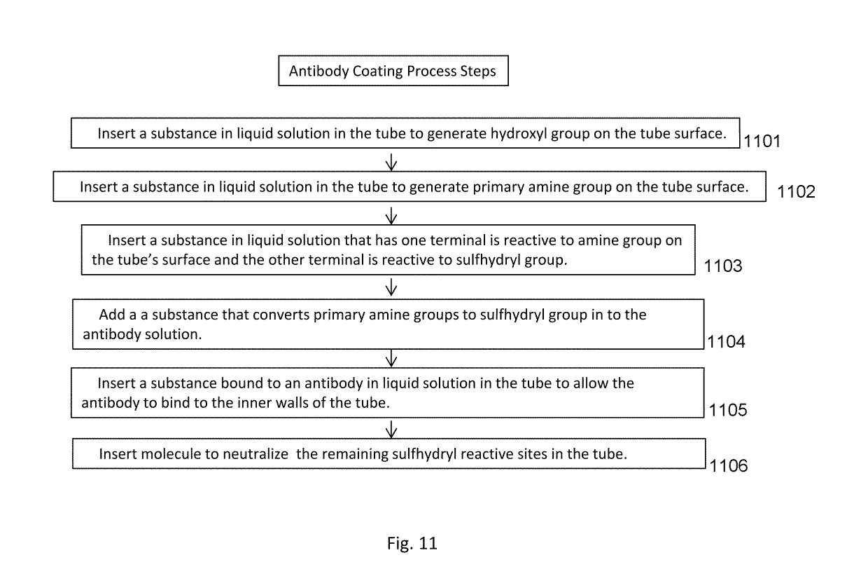

Turning now to FIG. 11, an exemplary process of applying the antibody coating to the tube described above may comprise the following steps: (1101) PDMS tube is treated by hydrogenperoxide (H2O2): hydrochloric acid (HCL): water (H2O) mixture. This treatment can generate hydroxyl group (—OH) on the PDMS tube inner surface. (1102) The tube is treated by aminopropyltrimethoxysilane (TMOS) (or aminopropyltriethoxyxilane (TEOS)). This step can produce primary amine group on the tube surface. (1103) The tube is filled with Sulfosuccinimidyl-4-(N-maleimidomethyl)cyclohexane-1-carboxylate (Sulfo-SMCC) solution (in buffer at pH 7.4). Sulfo-SMCC is a hetero-bifunctional-crosslinker (one terminal is reactive to amine group and the other terminal is reactive to sulfhydryl group). (1104) At the same time, 2-iminothiolane (2-IT) is added to antibody solution and the mixture is stirred at room temperature in a vial (not inside the tube yet). 2-IT converts primary amine groups in the given antibody to sulfhydryl group (—SH). Then, the excess 2-IT is removed from antibody solution by centrifugal filtration and the excess Sulfo-SMCC is removed from the tube (excess Sulfo-SMCC is defined as the Sulfo-SMCC that is unbound to the tube). (1105) Product from step 3-b, which is the antibody solution, is injected in the tube following step 3 a (in step 3 a the tube have been treated with Sulfo-SMCC). This step allows the sulfhydryl group on the antibody to react with sulfhydryl reactive terminal of sulfo-SMCC, resulting in antibody coated tube inner surface by covalent linkage. (1106) The antibody conjugated tube surface is treated by cystein solution. Cystein (an amino acid with —SH group) can cap the remaining sulfhydryl reactive site of tube and neutralize the electric charge of the tube surface. One of ordinary skill in the art would appreciate that there a number of modifications that could be made to the above described steps without departing from spirit and scope of the present invention.

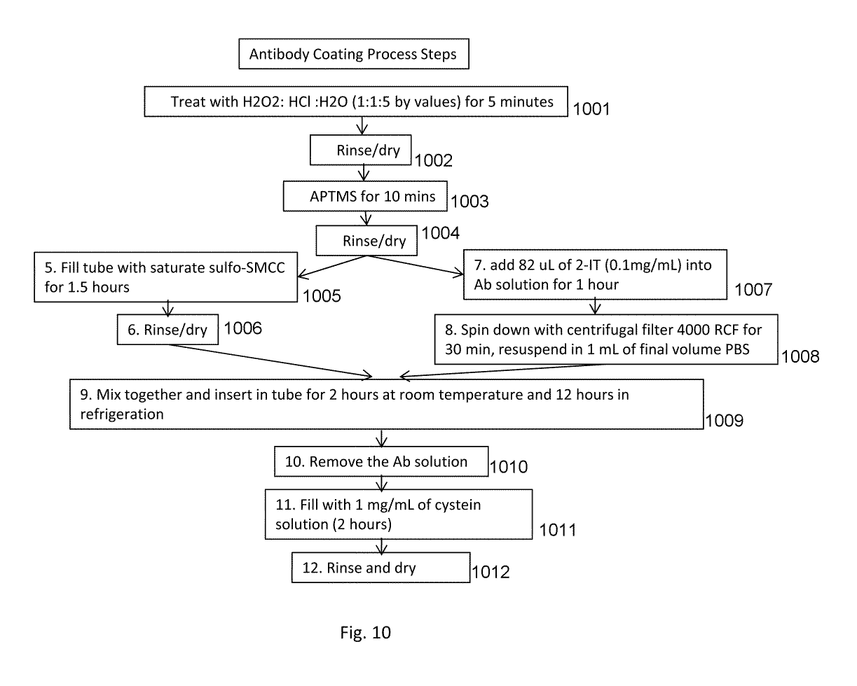

According to an embodiment of the present invention, a polydimethylsiloxane (PDMS) tubing (laboratory tubing with 1.02 mm in inner diameter) can be used (FIG. 1 (A)). The tube's internal surface is activated by treating with acidic hydrogenperoxide solution (H₂O:HCl:H₂O₂ in 5:1:1 volume ratio) for 5 minutes at room temperature (FIG. 10 step 1001). The tube is rinsed with excess deionized (DI) water 5 times and dried in air (FIG. 10 step 1002). This treatment forms the hydrophilic surface with hydroxyl groups available for further functionalization. Then, the tube is filled with aminopropyltrimethoxysilane (APTMS) for 10 minutes (FIG. 10 step 1003). The tube is rinsed with excess amount of DI water at least 5 times and dried in air. This step adds the primary amine group on the surface based on the sol-gel reaction principle (FIG. 10 step 1004). Then, the tube is rinsed and the fluorescence from tube's inner surface is monitored using fluorescence microscope.

EpCAM is a widely accepted CTC marker due to CTC's epithelial origin. Therefore, according to an embodiment of the present invention, EpCAM antibody is treated with Traut's reagent (2-iminothiolane HCl, 2-IT) to generate an available sulfhydryl group (—SH) (anti-EpCAM:2-IT=1:10 in mole ratio) in PBS (pH 7.4) for 1 hour (FIG. 10 step 1007). Then, unbound 2-IT is removed from the antibodies using centrifugal filter (MWCO 30 kDa, Amicon filter or Corning Spin-X protein concentrator) at 4000 RCF for 30 minutes (FIG. 10 step 1008). The concentrated anti-EpCAM is resuspended in PBS, adjusting the volume of 1 mL. During the antibody-2-IT reaction, the amine functionalized tube is filled with a hetero-bifunctional (amine reactive at one terminal and thiol reactive at the other terminal) cross-linker, sulfo-SMCC (sulfosuccinimidyl 4-[N-maleimidomethyl]cyclohexane-1-carboxylate) in 2 mg/mL concentration in PBS (pH 7.4) (FIG. 10 step 1005). After the EpCAM is spinned down, the sulfo-SMCC solution is removed from tube, and the tube is rinsed in PBS and re-filled with 1 mL EpCAM solution (FIG. 10 step 1006). The reaction is run for 2 hours at room temperature and kept on going overnight at 4° C. on a shaker (FIG. 10 step 1009). The next day, after the unbound EpCAM solution is collected (FIG. 10 step 10), the tube is gently rinsed with PBS and then refilled with 1 mg/mL L-cystein for further 2 hours (FIG. 10 step 1011). The tube is rinsed and dried (FIG. 10 step 1012). The conjugation of anti-EpCAM on the tube surface is confirmed by PE's fluorescence on a fluorescence microscope. One of ordinary skill in the art would appreciate that there a number of modifications that could be made to the above described steps without departing from spirit and scope of the present invention.

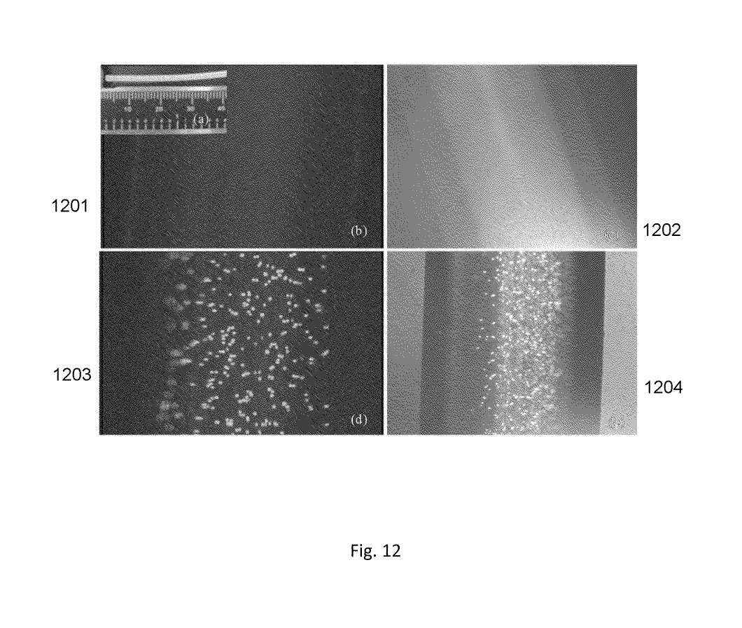

Turning now to FIG. 12, at element 1201 (a) a tube, like the one shown in the picture, are functionalized with human anti-EpCAM (ruler scale in mm) as described above. As shown in 1201 and 1202, PC-3 cells were placed in an unmodified tube (without EpCAM coating), for control measurements, no capture was observed. As shown in 1203 and 1204, fluorescent microscopic images of captured PC-3 cells on anti-EpCAM immobilized tube (light areas shown in the tubes). The images in 1203 and 1204 are of captured PC-3 cells by anti-EpCAM conjugated silicone (PDMS) tube after 1 hour of incubation. After collecting the solution from tube, captured cells were stained with Calcein AM containing cell media and imaged using GFP filter cube (Ex: 485 nm/Em: 525 nm) with an Olympus IMT-2 fluorescence microscope. The result showed that PC-3 cells were effectively captured by the anti-EpCAM immobilized tube. Due to the fact that Calcein AM is a cell viability indicating fluorescent probe, these images also confirm that the captured cells are alive. In contrast the unmodified control tubes, shown in 1201 and 1202, exhibited negligible capture of PC-3 cells.

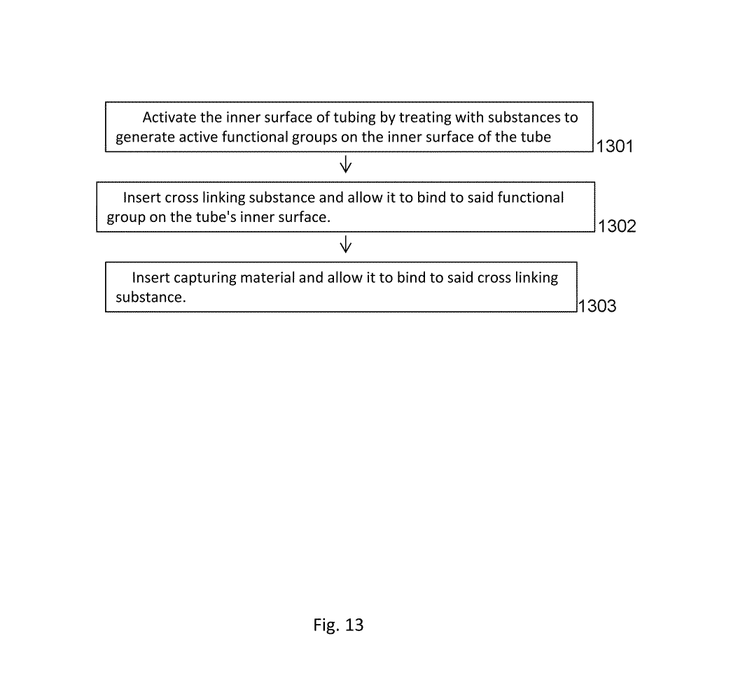

Turning now to FIG. 13, an exemplary process to functionalize a tube for capturing specific substances may comprise the following steps: (1301) activate the inner surface of tubing by treating with substances to generate active functional groups on the inner surface of the tube; (1302) insert cross linking substance and allow it to bind to said functional group on the tube's inner surface; (1303) insert capturing material and allow it to bind to said cross linking substance. Said capturing material is designed to bind to the specific substance. According to an embodiment of the present invention substances to generate active functional groups are selected from the group of active functional group generating substances comprising acidic hydrogenperoxide solution (H₂O:HCl:H₂O₂ in 5:1:1 volume ratio), aminopropyltrimethoxysilane (APTMS). According to an embodiment of the present invention cross linking substances are selected from the group of cross linking substance comprising 1-Ethyl-3-[3-dimethylaminopropyl]carbodiimide hydrochloride (EDC or EDAC), sulfo-SMCC (sulfosuccinimidyl 4-[N-maleimidomethyl]cyclohexane-1-carboxylate), polymer, polymeric linker, Polyethylene Glycol (PEG). According to an embodiment of the present invention capturing materials are selected from the group of capturing material comprising antibodies, aptamers, peptides, polymers, proteins, nucleic acid, RNA, DNA, organic materials, magnetic particles. Said disease causing material is selected from the group comprising cancer cells, circulating tumor cells, bacteria, virus, fungi, toxic materials, peptides, proteins, molecules, mesenchymal tumor cells, cancer stem cells, cholesterol, beta amyloid, circulating tumor DNA. One of ordinary skill in the art would appreciate that there are numerous types of disease causing material that could be captured through use of embodiments of the present invention, and embodiments of the present invention are contemplated for use in capturing any appropriate type of disease causing material.

According to an embodiment of the present invention, the tube is a medical tube. In a preferred embodiment, the tube is selected from a group of tube comprising plastic tubes, polymer tube, metallic tube, silicone tube. In one embodiment, the captured cells on the tube are counted and further re-suspended and genetically analyzed. In another embodiment, additional filters and apoptosis causing agents are added to enhance the capture/kill rate. In another embodiment, this method can be applied to other conditions requiring blood cleansing, for example sepsis, poisoning, leukemia, cholesterols and so on. In another embodiment, the system is part a dialysis machine. In another embodiment, a machine that includes the tube also includes anticoagulant inlets, filters to filter cells by size (for example 25 um size separation holes), and photodynamic therapy.

While the invention has been thus described with reference to the embodiments, it will be readily understood by those skilled in the art that equivalents may be substituted for the various elements and modifications made without departing from the spirit and scope of the invention. It is to be understood that all technical and scientific terms used in the present invention have the same meaning as commonly understood by one of ordinary skill in the art to which this invention belongs. Accordingly, the drawings and descriptions are to be regarded as illustrative in nature and not restrictive.

1. A method for preparing a tube to be used for capturing disease causing material, said method comprising the steps of:
activating an inner surface of the tube by treating the inner surface with substances to generate active functional groups on the inner surface of the tube;
inserting into the tube a crosslinking substance such that the crosslinking substance binds to said functional group on the inner surface of the tube;
inserting capturing material into the tube such that the capturing material binds to said crosslinking substance, wherein said capturing material is designed to bind to said substances.

2. The method of claim 1, wherein said tube is selected from a group comprising plastic tube, polymer tube, metallic tube and silicone tube.

3. The method of claim 1, wherein said substance to generate active functional groups is selected from the group comprising acidic hydrogenperoxide solution (H₂O:HCl:H₂O₂ in 5:1:1 volume ratio) and aminopropyltrimethoxysilane (APTMS).

4. The method of claim 1, wherein said crosslinking substance is selected from the group comprising 1-Ethyl-3-[3-dimethylaminopropyl]carbodiimide hydrochloride (EDC or EDAC), sulfo-SMCC (sulfosuccinimidyl 4-[N-maleimidomethyl]cyclohexane-1-carboxylate), polymer, polymeric linker and Polyethylene Glycol (PEG).

5. The method of claim 1, wherein said capturing material is selected from the group comprising antibodies, aptamers, peptides, polymers, proteins, nucleic acid, RNA, DNA, organic materials and magnetic particles.

6. The method of claim 1, wherein said disease causing material is selected from the group comprising cancer cells, circulating tumor cells, bacteria, virus, fungus, toxic materials, peptides, proteins, molecules, mesenchymal tumor cells, cancer stem cells.

7. A method for preparing a tube to be used for capturing disease causing material, said method comprising the steps of:
activating an internal surface of the tube by treating the internal surface with an acidic hydrogenperoxide solution to form a hydrophilic surface with hydroxyl groups;
filling the tube with aminopropyltrimethoxysilane to add a primary amine group on the internal surface;
treating an antibody with a solution to generate available sulfhydryl group (—SH);
filling the tube with a hetero-bifunctional cross-linker;
removing the excess hetero-bifunctional cross-linker solution from tube;
filling the tube with the antibody solution; and
filling the tube with L-cystein.
